(19) United States
(12) Patent Application Publication
(10) Pub. No.: US 2022/0031237 A1
(43) Pub. Date: Feb. 3, 2022

(54) SYSTEMS, METHODS AND COMPUTER PROGRAM PRODUCT FOR MONITORING VASCULAR PERFUSION IN REPLANTED TISSUE FLAPS

(71) Applicant: Secretary, Department of Biotechnology, New Delhi (IN)

(72) Inventors: Nirmal Kumar, District Bharaich (IN); Aniket Anand Kulkarni, Pune (IN); Deepika Dixit, Pune (IN); Yasuyuki Matsuura, Hiroshima (JP); Prashant Jha, New Delhi (IN); Ashish Bichpuriya, Nagpur (IN)

(21) Appl. No.: 17/299,628

(22) PCT Filed: Dec. 2, 2019
(86) PCT No.: PCT/IB2019/060358

§ 371 Date: Jun. 3, 2021

(30) Foreign Application Priority Data
Dec. 3, 2018 (IN) 201811045670

(51) Int. Cl. A61B 5/00; A61B 5/0205; G16H 40/63

(52) U.S. Cl. CPC A61B 5/4848; A61B 5/02055; A61B 5/024; G16H 40/63; A61B 5/7425

(57) ABSTRACT

The invention provides systems, methods and computer program products for monitoring vascular perfusion in replanted tissue flaps. Specifically, the invention provides a non-invasive solution for monitoring of vascular perfusion at a site of tissue replantation, and that is capable of detecting problems in vascular perfusion and raising alarms in real times. The invention achieves the above function objectives by means of non-invasive sensors that continuously monitor selected parameters related to tissue condition. The monitored data parameters are used to determine a real time condition of replanted tissue.

### CROSS-REFERENCE TO RELATED APPLICATIONS

This Application is a National Stage Application under 35 U.S.C. §371 of PCT Application No. PCT/IB2019/060358, filed Dec. 2, 2019, which claims priority from and the benefit of Indian Patent Application No. 201811045670 filed on Dec. 3, 2018, which are hereby incorporated by reference in their respective entireties.

### FIELD OF THE INVENTION

The present invention relates to the domain of post-operative monitoring and health care. In particular, the invention provides improved systems, methods and computer program products for monitoring vascular perfusion in replanted tissue flaps.

### BACKGROUND

Flap surgery comprises a type of tissue reconstructive procedure involving removal and transplanting of a flap of tissue from one area of a body to another. The transplanted or replanted flap comprises a section of living tissue with a blood supply that may be transported from a “donor” area of a body to a new area of the body. A flap may be transplanted or replanted to an area of the body that has lost skin, fat or muscle.

Flap replantation typically requires that the replanted flap receive blood supply from blood vessels. For this reason, the tissue flap is surgically severed from blood vessels at the donor site and the severed ends of the tissue flap are reconnected or attached to blood vessels at the recipient site—which ensures a continuing blood supply and oxygen supply to the replanted flap. FIG. 1 is a photograph of a subject's limb 100 to which a tissue flap 102 has been surgically replanted using sutures 104.

As a result of the surgical replanting of tissue and reattachment of blood vessels, monitoring blood circulation and vascular perfusion at a site of tissue replantation is critical to post-operative care—since inadequate perfusion need to be recognized quickly to correct any treatable problems. Failure to monitor changes in perfusion and correct any problems can result in partial or complete tissue loss associated with the replanted flap. The main reasons for free flap failure, have been found to be vascular thrombosis, arterial insufficiency, active bleeding or hematoma and/or venous congestion.

Although there are numerous techniques to assess flap vitality, clinical examination remains the most commonly used one. Health care providers charged with this responsibility may often not have the necessary experience to assess the state of the flap. Even young plastic surgeons often admit to uncertainty in assessing post-operative flap vitality.

Another solution type in the state of art consist of periodically drawing blood from both the replanted tissue and a region of healthy tissue. Both samples are subject to blood glucose measurement, and any significant difference in blood glucose measured at the tissue replantation site in comparison with the blood glucose measured at the healthy tissue site is treated as an indication of a problem or a blockage in vascular perfusion. The problem would then be further investigated using Doppler or ultrasound techniques.

The prior art solutions suffer from significant drawbacks including being invasive, subjecting the replanted tissue to additional trauma during blood sample collection, and only being able to indicate problems in response to the periodic collection and testing of blood samples—which eliminates the possibility of real time monitoring.

There is accordingly a need for a solution that enables real time, non-invasive monitoring of vascular perfusion at a site of tissue replantation, and that is capable of detecting problems in vascular perfusion and raising alarms in real time.

### BTIEF DESCRIPTION OF THE ACCOMPANYING DRAWINGS

FIG. 1 illustrates a replanted tissue flap on a limb.

FIGS. 2A, 2B and 2C illustrate exemplary embodiments of sensor patches configured in accordance with the teachings of the present invention.

FIGS. 2D and 2E illustrate exemplary embodiments of a housing having a plurality of sensors, configured in accordance with the teachings of the present invention.

FIGS. 3, 4 and 4A illustrate exemplary embodiments of system environments configured to implement teachings of the present invention.

FIGS. 5, 6 and 10 to 15 are flowcharts illustrating exemplary methods in accordance with the teachings of the present invention.

FIGS. 7 to 9 illustrate exemplary decision factors in raising alarms in the system environments after present invention.

FIG. 16 illustrates a graph corresponding to the output of magnification implemented in accordance with the teachings of the present invention, based on a fixed baseline value.

FIG. 17 illustrates a graph corresponding to the output of magnification implemented in accordance with the teachings of the present invention, based on a variable baseline value.

FIG. 18 illustrates a graph corresponding to the output of color magnification implemented in accordance with the teachings of the present invention.

FIG. 19 illustrates an exemplary computer system that may be configured to implement the teachings of the present invention.

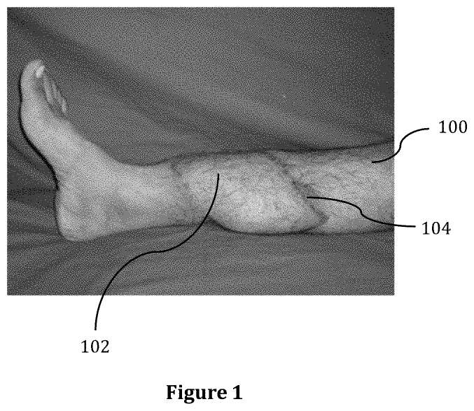

### SUMMARY

The present invention relates to the domain of post-operative monitoring and health care. In particular, the invention provides improved systems, methods and computer program products for monitoring vascular perfusion in replanted tissue flaps.

In an embodiment, the invention provides an apparatus for non-invasive monitoring of vascular perfusion at a site of tissue replantation, comprising (i) a processor, (ii) a transceiver, and (iii) one or more of a first temperature sensor, a first SpO₂ sensor, a first pulse sensor and a first color sensor.

The processor may be configured to (i) receive from one or more of a first temperature sensor, a first SpO₂ sensor, a first pulse sensor and a first color sensor respectively, one or more of temperature data, SpO₂ data, pulse data and color data corresponding to a tissue replantation site; and (ii) generate an alarm in response to determining, based on any of the received temperature data, SpO₂ data, pulse data and color data received, that any of a detected temperature parameter value, SpO₂ parameter value, pulse parameter value and color parameter value corresponding to the tissue replantation site, is inconsistent with one or more normal values for temperature data, SpO₂ data, pulse data or color data.

The apparatus may be configured such that the one or more normal values for temperature data, SpO₂ data, pulse data or color data comprise a range of predefined normal values associated with healthy tissue.

The apparatus may be configured such that the one or more normal values for temperature data, SpO₂ data, pulse data or color data comprise one or more of temperature data values received from a second temperature sensor, SpO₂ data received from a second SpO₂ sensor, pulse data received from a second pulse sensor and color data received from a second color sensor, wherein any of the second temperature sensor, second SpO₂ sensor, second pulse sensor, and second color sensor are configured to detect data values corresponding to a healthy tissue site.

In an embodiment, the apparatus may comprise all of a first temperature sensor, a first SpO₂ sensor, a first pulse sensor and a first color sensor.

The apparatus may be configured such that the alarm is generated based on an evaluation of two or more of temperature data, SpO₂ data, pulse data and color data corresponding to the tissue replantation site.

In a particular embodiment, the apparatus may be configured such that the alarm is generated based on an evaluation of all of temperature data, SpO₂ data, pulse data and color data corresponding to the tissue replantation site.

The apparatus may be configured such that data received from any of the first temperature sensor, first SpO₂ sensor, first pulse sensor and first color sensor respectively is magnified for display, wherein a magnified output is determined as:

Magnified Output=(((Sensor Output)−(Baseline Output))×(Magnification Factor))+(Baseline Value).

The invention additionally provides a method for non-invasive monitoring of vascular perfusion at a site of tissue replantation, comprising implementing at a processor, the steps of (i) receiving from one or more of a first temperature sensor, a first SpO₂ sensor, a first pulse sensor and a first color sensor respectively, one or more of temperature data, SpO₂ data, pulse data and color data corresponding to a tissue replantation site, and generating an alarm in response to determining, based on any of the received temperature data, SpO₂ data, pulse data and color data received, that any of a detected temperature parameter value, SpO₂ parameter value, pulse parameter value and color parameter value corresponding to the tissue replantation site, is inconsistent with one or more normal values for temperature data, SpO₂ data, pulse data or color data.

In a method embodiment, the one or more normal values for temperature data, SpO₂ data, pulse data or color data, comprise a range of predefined normal values associated with healthy tissue.

The one or more normal values for temperature data, SpO₂ data, pulse data or color data comprise one or more of temperature data values received from a second temperature sensor, SpO₂ data received from a second SpO₂ sensor, pulse data received from a second pulse sensor and color data received from a second color sensor, wherein any of the second temperature sensor, second SpO₂ sensor, second pulse sensor, and second color sensor are configured to detect data values corresponding to a healthy tissue site.

In an embodiment, the method comprises receiving all of temperature data, SpO₂ data, pulse data and color data corresponding to a tissue replantation site.

In another method embodiment, the alarm is generated based on an evaluation of two or more of temperature data, SpO₂ data, pulse data and color data corresponding to the tissue replantation site. The alarm may in an embodiment be generated based on an evaluation of all of temperature data, SpO₂ data, pulse data and color data corresponding to the tissue replantation site.

In a particular embodiment of the method, data received from any of the first temperature sensor, first SpO₂ sensor, first pulse sensor and first color sensor respectively is magnified for display, wherein a magnified output is determined as:

Magnified Output=(((Sensor Output)−(Baseline Output))×(Magnification Factor))+(Baseline Value).

The invention additionally presents a computer program product for non-invasive monitoring of vascular perfusion at a site of tissue replantation, comprising a non-transitory computer readable medium having computer readable instructions for implementing the steps of (i) receiving from one or more of a first temperature sensor, a first SpO₂ sensor, a first pulse sensor and a first color sensor respectively, one or more of temperature data, SpO₂ data, pulse data and color data corresponding to a tissue replantation site, and (ii) generating an alarm in response to determining, based on any of the received temperature data, SpO₂ data, pulse data and color data received, that any of a detected temperature parameter value, SpO₂ parameter value, pulse parameter value and color parameter value corresponding to the tissue replantation site, is inconsistent with one or more normal values for temperature data, SpO₂ data, pulse data or color data.

### DETAILED DESCRIPTION

The present invention provides systems, methods and computer program products for monitoring vascular perfusion in replanted tissue flaps. Specifically, the invention provides a non-invasive solution for monitoring of vascular perfusion at a site of tissue replantation, and that is capable of detecting problems in vascular perfusion and raising alarms in real time.

The invention achieves the above objectives by means of non-invasive sensors that continuously monitor selected parameters related to tissue condition. The monitored data parameters are used to determine a real-time condition of replanted tissue.

It would be understood that based on the teachings hereinbelow, any number of different sensors may be used for the present invention. Exemplary sensors that may be configured in accordance of the present invention include:

Color Sensor: To monitor the RGB (Red, Green, Blue) values of the flap. In case of interrupted perfusion, the flap tends to lose its color (drop in red color)

Temperature Sensor: This sensor monitors flap temperature.

SpO2: This sensor measures the blood oxygen content. It has been found that oxygen content tends to drop in case of vascular/arterial blockage.

Pulse: This sensor measures the variation in superficial veins due to pulsation.

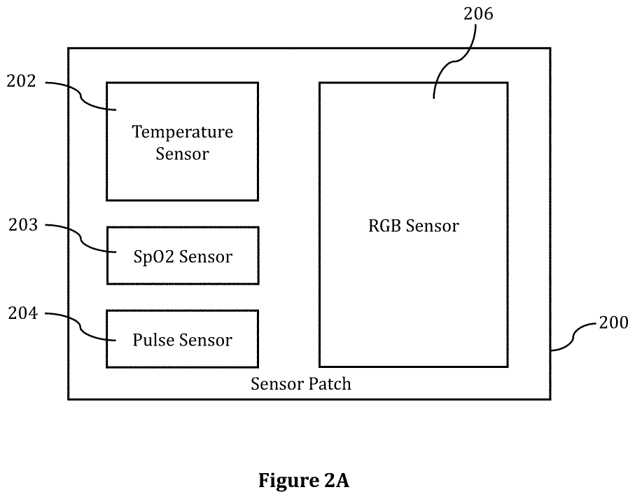

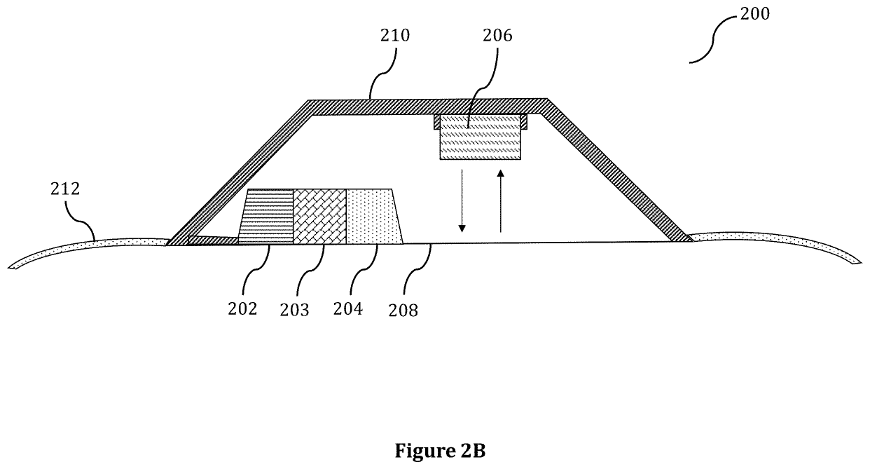

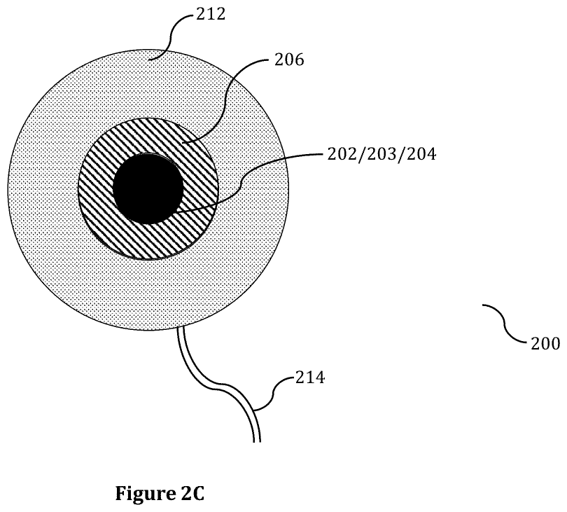

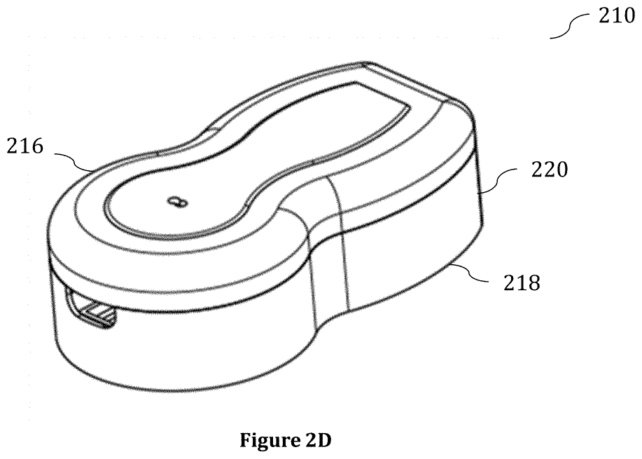

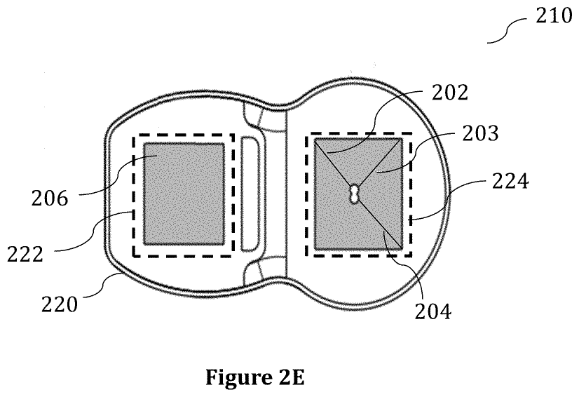

FIG. 2A illustrates a block diagram showing components within a sensor patch 200 of a kind that can be used for monitoring of vascular perfusion at a tissue replantation site. As illustrated in FIG. 2, sensor patch 200 includes temperature sensor 202, SpO2 sensor 203, pulse sensor 204 and a color sensor/RGB sensor 206.

FIG. 2B illustrates a cross sectional view of sensor patch 200 that has been applied to a replanted tissue site 208. As shown in FIG. 2B, sensor patch 200 includes an adhesive substrate 212 that enables the patch to be affixed to a subject's skin at or around a replanted tissue site.

Sensor patch 200 additionally includes a housing 210 that is affixed to or integrated with adhesive substrate 212. Said housing 210 includes temperature sensor 202, SpO2 sensor 203, pulse sensor 204 and color/RGB sensor 206 housed therewithin.

Housing 210 is configured such that by affixing adhesive substrate 212 at or around a replanted tissue site 208, the sensors 202, 203, 204 and 206 are positioned so as to be able to respectively generate temperature readings, pulse and SpO2 readings and color/RGB sensor readings corresponding to the replanted flap.

FIG. 2C illustrates a plan view of sensor patch 200, and illustrates an exemplary arrangement of adhesive substrate 212, and sensors 202, 203, 204 and 206. Additionally, sensor patch 200 may include a wire or cable 214 for transmitting electrical signals or data signals between sensors 202 to 206 and a processing unit (not shown) that is communicably coupled with sensor patch 200. It would be understood that in other embodiments, sensors 202 to 206 may communicate with the processing unit wirelessly through a transceiver (not shown) located within sensor patch 200.

FIGS. 2D and 2E illustrate an alternative embodiment of housing 210 having sensors 202, 203, 204 and 206 disposed therein.

FIG. 2D illustrates a perspective view of housing 210 may comprise an enclosure having a top surface 216, a bottom surface 218 and a sidewall surface 220 coupling top surface 216 and bottom surface 218. Top surface 216, bottom surface 218 and sidewall surface 220 together define an interior volume or interior chamber configured to house one or more of temperature sensor 202, SpO₂ sensor 203, pulse sensor 204 and color/RGB sensor 206.

FIG. 2E illustrates a bottom plan view of housing 210. As shown in FIG. 2E, bottom surface 218 may comprise a plurality of apertures 222 and 224. Sensors 202, 203, 204 and 206 are positioned within the interior volume or interior chamber of housing 210 such that said sensors can interface with a tissue flap through one or more of apertures 222 and 224 and can respectively generate temperature readings, pulse and SpO2 readings and color/RGB sensor readings corresponding to the replanted flap.

In operation, the housing 210 of FIG. 2D and 2E may be placed such that bottom surface 218 of housing 210 is placed against or facing the replanted tissue flap—such that sensors 202, 203, 204 and 206 can respectively generate temperature readings, pulse and SpO2 readings and color/RGB sensor readings corresponding to the replanted flap through apertures 222 and 224.

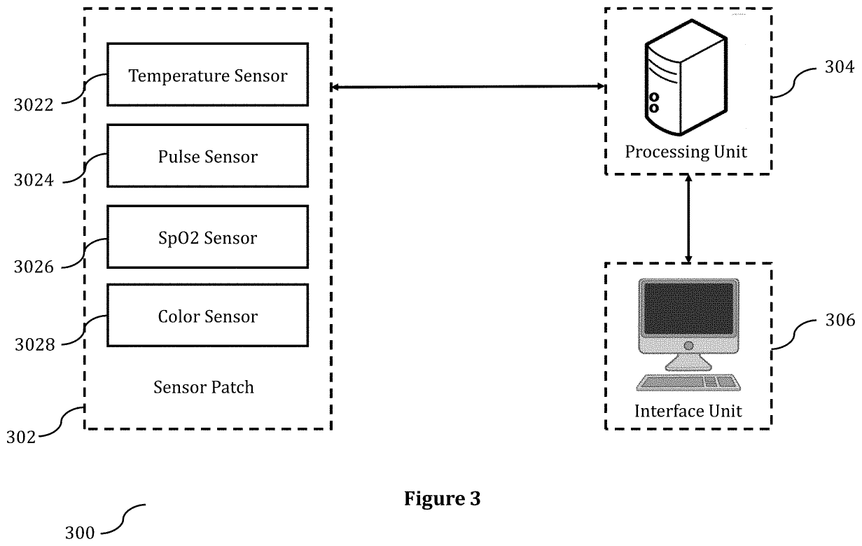

FIG. 3 illustrates a first system environment 300 within which the teachings of the present invention may be implemented. As shown in FIG. 3, sensor patch 300 may include temperature sensor 3022, pulse sensor 3024, SpO2 sensor 3026 and color sensor 3028. Sensor patch 300 may be communicably coupled (either wirelessly or through a wire based medium) with processing unit 304. Processing unit 304 may comprise any processor based signal processing and signal analysis unit that is configured to process and analyze signals received from sensor patch 200 and to monitor the state of a replanted tissue flap based on the received signals and based on one or more predefined monitoring and analysis rules.

As discussed in more detail hereinbelow, the data received from sensors within sensor patch 300 located at a tissue replantation site may be compared against predefined baselines values or against data received from sensors within one or more other sensor patches located at healthy tissue sites, so that any significant differences between data corresponding to the tissue replantation site in comparison with the predefined baseline values or in comparison with data received from sensor patches applied to healthy tissue sites, can be used to identify abnormalities in vascular perfusion.

Parameter data and/or alarms may be communicated from processing unity 304 to a user, operator, healthcare provider or any other person through interface unit 306. Said interface unit may comprise any interface device capable of providing visual representations of data/alerts/alarms or of providing audio or tactile feedback as a representation of an alert or an alarm.

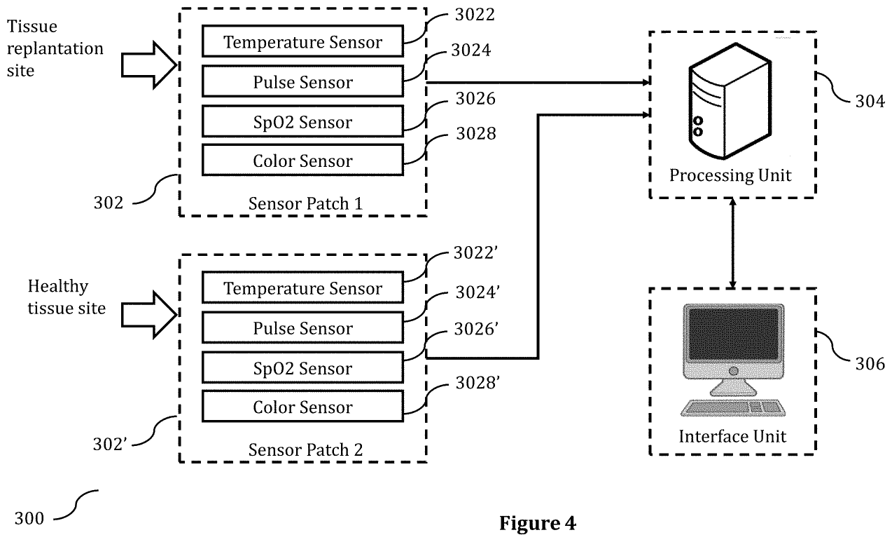

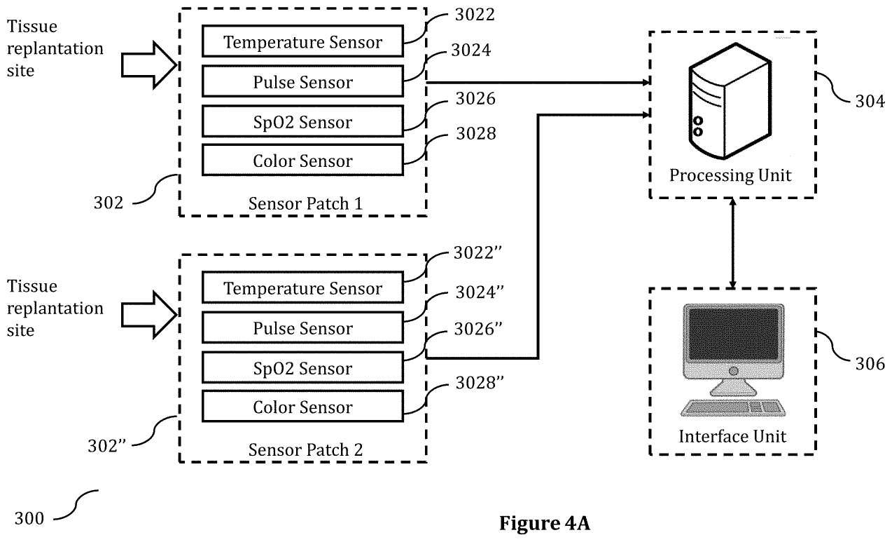

FIG. 4 illustrates another embodiment of system environment 300, wherein, in addition to a first sensor patch (sensor patch 1) 302, processing unit 304 and interface unit 306 that have been described in connection with FIG. 3, said system environment 300 additionally includes a second sensor patch (sensor patch 2) 302′ comprising a distinct second set of sensors including temperature sensor 3022′, pulse sensor 3024′, SpO2 sensor 3026′ and color sensor 3028′. The second sensor patch 302′ is also communicably coupled with processing unit 304.

By locating the first sensor patch 302 at a tissue replantation site and the second sensor patch 302′ at a site consisting of healthy tissue, processing unit 304 is enabled to receive data signals from both sites—so that said signals can be compared for the purposes of determining any abnormal event (i.e. problems in vascular perfusion) associated with said tissue replantation site.

FIG. 4A illustrates another embodiment of system environment 300, wherein, in addition to a first sensor patch (sensor patch 1) 302, processing unit 304 and interface unit 306 that have been described in connection with FIG. 3, said system environment 300 additionally includes a second sensor patch (sensor patch 2) 302″ comprising a distinct second set of sensors including temperature sensor 3022″, pulse sensor 3024″, SpO2 sensor 3026″ and color sensor 3028″. The second sensor patch 302″ is also communicably coupled with processing unit 304.

By locating the first sensor patch 302 at a first region of a tissue replantation site and the second sensor patch 302″ at a second region of said tissue replantation site, processing unit 304 is enabled to receive data signals from independent sensors/sensor patches located at two different regions within a tissue replantation site—so that said signals from both regions can be combined and/or assessed in a combined manner (based on one or more rules for combining sensor regions) so as to enable identification of any abnormal event (i.e. problems in vascular perfusion) associated with said tissue replantation site based the plurality of regions that are being monitored within said site. Obtaining sensor readings from multiple regions within the same tissue site has been found to improve accuracy of detection and identification of abnormal events, and also to reduce the probability of undue weightage being given to outlier readings that are generated as a consequence of any defect in a sensor or sensor patch, or as a consequence of improper application of a sensor or sensor patch to a tissue replantation site.

In other embodiments, the first sensor patch 302 and second sensor patch 302″ may be located respectively at a first tissue replantation site and at a second tissue replantation site—thereby enabling simultaneous monitoring of more than one tissue replantation site.

Methods by which the processing unit 304 monitors the state of a replanted tissue flap are described in more detail below.

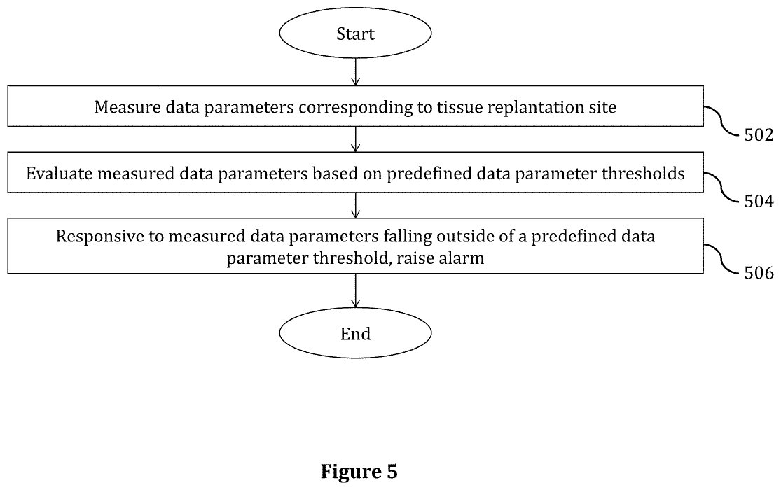

FIG. 5 illustrates a method of real time monitoring of vascular perfusion at a tissue replantation site. Step 502 comprises measuring data parameters corresponding to the site of tissue replantation/the replanted flap region. Said data parameters may comprise temperature data, pulse data, SpO2 data and/or color data received from sensor patches 302, 302′ or 302″. Step 504 comprises evaluating the measured data parameters based on one or more data parameter baselines or thresholds that have been defined in connection with such parameters—and which baselines or thresholds comprise the ranges of values that may be expected in the case of normal or acceptable vascular perfusion. At step 506, responsive to the measured data parameters falling outside the defined baselines or thresholds, alerts/alarms/indicators of abnormality may be generated with a view to raise an alert regarding the likelihood of abnormal or unacceptable vascular perfusion at the tissue replantation site.

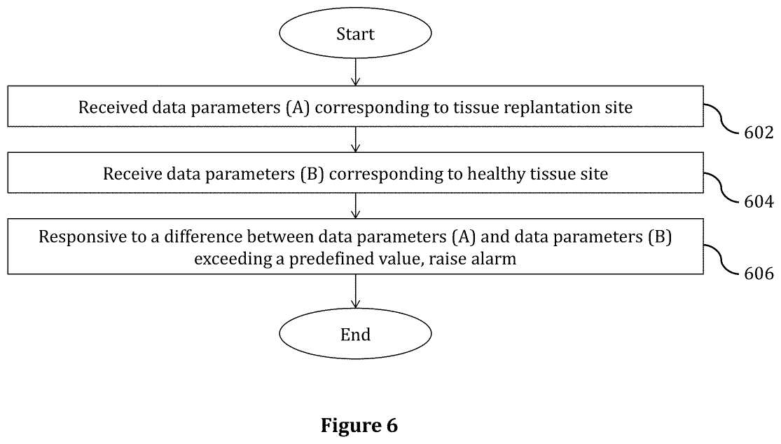

FIG. 6 illustrates a second method of real time monitoring of vascular perfusion at a tissue replantation site, using data received from a first sensor patch 302 monitoring data parameters at a tissue replantation site and using data received from a second sensor patch 302′ monitoring data parameters at a healthy tissue site.

Step 602 comprises measuring a first set of data parameters (A) corresponding to the site of tissue replantation/the replanted flap region. Said data parameters (A) may be generated based on sensor signals received a first sensor patch 302, and may comprise temperature data, pulse data, SpO2 data and/or color data received from sensor patch 302. Step 604 comprises measuring a second set of data parameters (B) corresponding to a site of healthy tissue. Said data parameters (B) may be generated based on sensor signals received a second sensor patch 302′, and may comprise temperature data, pulse data, SpO2 data and/or color data received from the second sensor patch 302′. The first set of data parameters (A) may be compared against the second set of data parameters (B) for identifying differences. At step 606, responsive to one or more of the first set of data parameters (A) being found to deviate or differ from one or more of the second set of data parameters (B) by more than a predefined value, baseline or range, an alert/alarm/indicator of abnormality is generated with a view to raise an alert regarding the likelihood of abnormal or unacceptable vascular perfusion at the tissue replantation site.

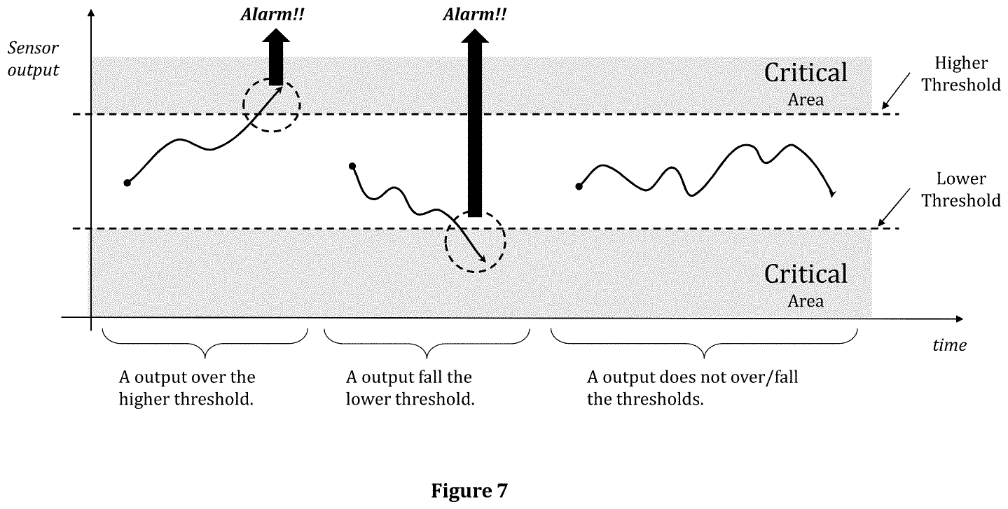

FIG. 7 illustrates a graph explaining some of the considerations based on which alarms may be raised to draw attention to events that are likely to indicate abnormal or unacceptable vascular perfusion when implementing the method of FIG. 5. As shown in FIG. 7 a predefined region of data parameter values may be defined such that sensor output above or below the predefined region of data parameter values may be considered to be an indicator of abnormal vascular perfusion at the tissue replantation site. If a sensor output comprises a value higher than the predefined highest acceptable value corresponding to the data parameter being measured by the sensor, an alarm may be raised. If a sensor output comprises a value lower than the predefined lowest acceptable value corresponding to the data parameter being measured by the sensor, an alarm may be raised. Sensor output falling within the range of predefined acceptable data parameter values corresponding to the data parameter being measured by the sensor are treated as being indicative of acceptable or normal vascular perfusion, and no alarm would be raised.

Both the highest acceptable value and lowest acceptable value may be changed dynamically based on the elapsed time from after a transplanted or replanted surgery, or based on the condition of tissue replantation sites.

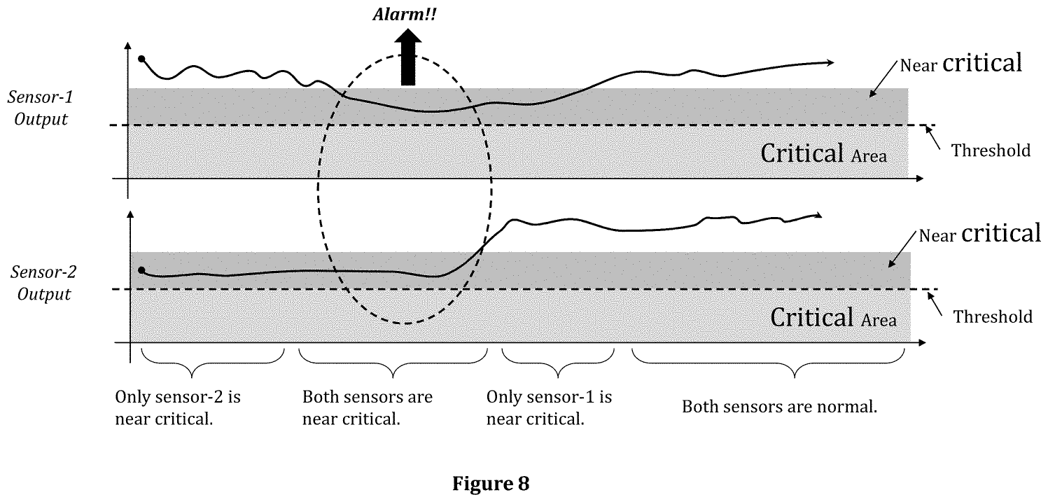

FIG. 8 illustrates a second graph explaining some of the considerations based on which alarms may be raised to draw attention to events that are likely to indicate abnormal or unacceptable vascular perfusion based on a combination of data received from multiple sensors.

As shown in FIG. 8, the graph represents real time data received from sensor 1 and from sensor 2, wherein sensor 1 is a first sensor located at a site of tissue replantation, and sensor 2 is a second sensor located at the site of tissue replantation. As shown in FIG. 8, each of sensor 1 and sensor 2 has a respective predefined range of data parameter values that are considered acceptable or indicative of normal vascular perfusion—and sensor outputs above or below the predefined range of acceptable data parameter values may be considered an indicator of abnormal vascular perfusion at the tissue replantation site.

If a sensor output comprises a value outside of the range of values defined as being acceptable for that sensor, such outlier value(s) may be treated as an indicator of abnormality. The invention may thereafter combine the outputs of the two sensors according to one or more rules for combination to determine whether the combined output should be treated as an event to trigger an alarm or alert. For example, in the graph of FIG. 8, an alarm is only raised if data parameters corresponding to both sensor 1 and sensor 2 are outside their respective predefined ranges of acceptable data values. If on the other hand only one of the two sensors outputs an outlier data parameter value, or neither of the two sensors outputs an outlier data parameter value, no alarm would be raised. In an embodiment of the invention, sensor 1 and sensor 2 are each configured to measure different parameters from the other.

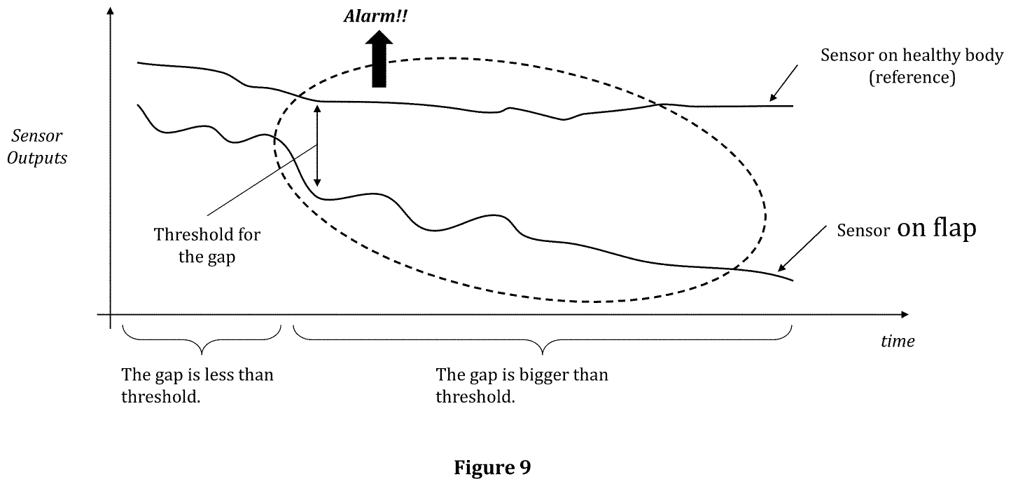

FIG. 9 illustrates a third graph explaining some of the considerations based on which alarms may be raised to draw attention to events that are likely to indicate abnormal or unacceptable vascular perfusion when implementing the method of FIG. 6.

As shown in FIG. 9, the graph represents real time data received from two sensors, wherein a first sensor provides data parameters corresponding to a site of tissue replantation and a second sensor provides data parameters corresponding to a healthy tissue site. In an embodiment, each of the first sensor and the second sensor are configured to measure the same data parameter. In the event a deviation or difference between the data parameter values received from the first sensor (i.e. corresponding to the site of tissue replantation) and the data parameter values received from the second sensor (i.e. corresponding to the site of healthy tissue) at substantially the same time, exceeds a defined threshold value, said difference or deviation may be treated as an indicator of abnormality, and an alarm may be raised.

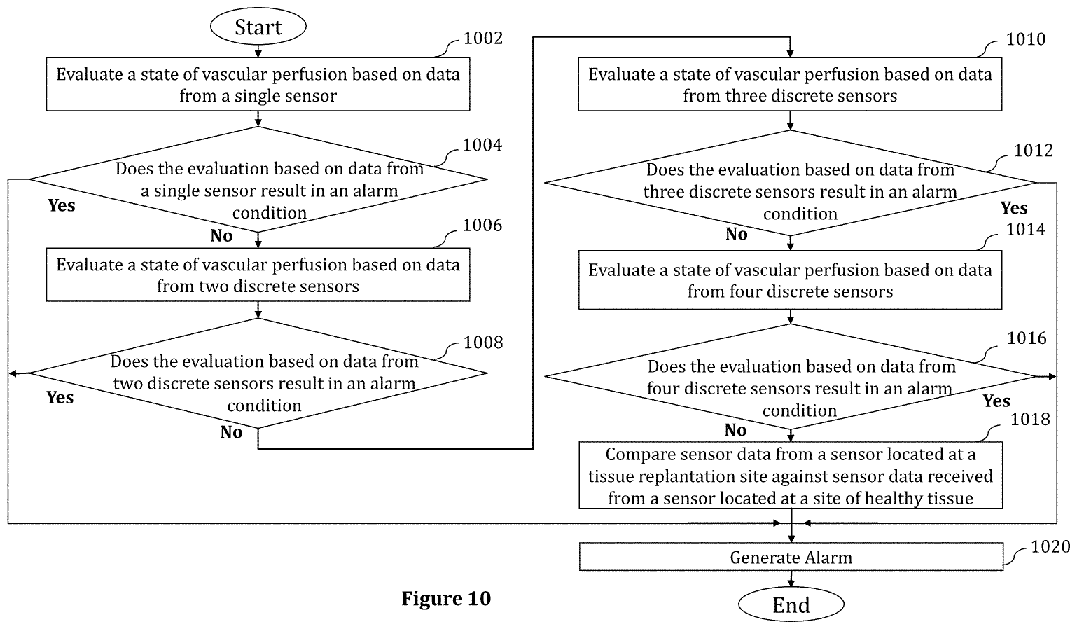

FIG. 10 illustrates a method according to the present invention. The method comprises method steps 1002 up to 1020 which are briefly described below:
Step 1002—Evaluate a state of vascular perfusion based on data from a single sensor
Step 1004—Does the evaluation based on data from a single sensor result in an alarm condition (if yes, proceed to raise an alarm at step 1020 and the method ends, and if no, proceed to step 1006)
Step 1006—Evaluate a state of vascular perfusion based on data from two discrete sensors
Step 1008—Does the evaluation based on data from two discrete sensors result in an alarm condition (if yes, proceed to raise an alarm at step 1020 and the method ends, and if no, proceed to step 1010)
Step 1010—Evaluate a state of vascular perfusion based on data from three discrete sensors
Step 1012—Does the evaluation based on data from three discrete sensors result in an alarm condition (if yes, proceed to raise an alarm at step 1020 and the method ends, and if no, proceed to step 1010)
Step 1014—Evaluate a state of vascular perfusion based on data from four discrete sensors
Step 1016—Does the evaluation based on data from four discrete sensors result in an alarm condition (if no, proceed to raise an alarm at step 1020 and the method ends, and if no, proceed to step 1018)
Step 1018—Compare sensor data from a sensor located at a tissue replantation site against sensor data received from a sensor located at a site of healthy tissue—and use the output of such comparison as the basis for a decision on whether to generate an alarm at step 1020.

Stated differently, the method of FIG. 10 involves a processing unit first checking the data from each individual sensor at the site of tissue replantation for indicators of abnormality (for example in accordance with the method of FIG. 5 and the graph of FIG. 7)—and raising an alert or alarm if any such abnormality is detected. If no abnormality is detected, the data processing unit iteratively progresses through combinations of data from increasingly larger number of sensors (e.g. combinations of data from 2 sensors, 3 sensors and 4 sensors) located at the site of tissue replantation (for example in accordance with the method discussed in connection with the graph of FIG. 8)—and raises an alert or alarm if any abnormality is detected based on the combination of data parameter values received from the multiple sensors. Lastly, the method compares the data values received from the sensors positioned at the site of tissue replantation against data values received from sensors positioned at a site of healthy tissue (for example in accordance with the methods discussed in connection with the flowchart of FIG. 6 and the graph of FIG. 9)—and raises an alert or alarm if any detected difference between the sensors at the site of tissue replantation and the sensors positioned at a site of healthy tissue exceeds a predefined threshold value.

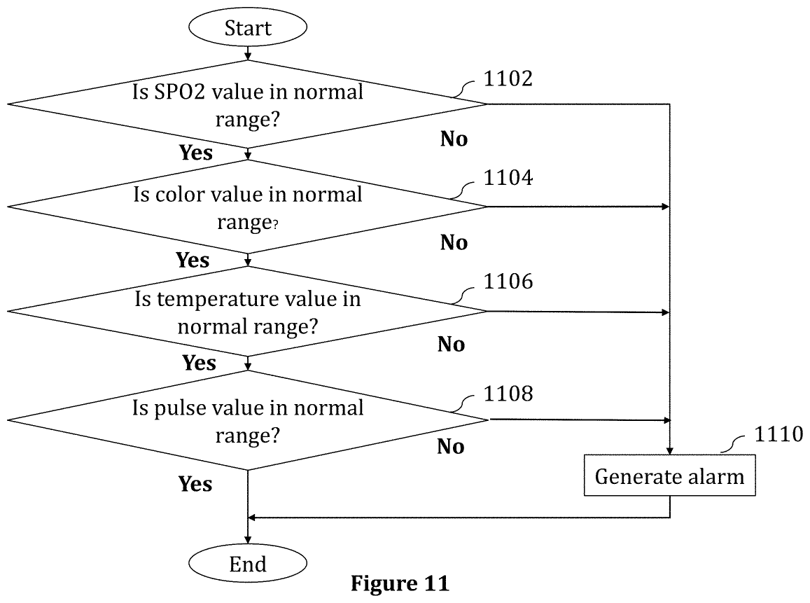

FIG. 11 illustrates a method of a detecting abnormal vascular perfusion based on data received from sensor patch 302 (i.e. comprising an SpO2 sensor, a color sensor, a temperature sensor and a pulse sensor) in accordance with the method of FIG. 5. The method comprises method steps 1102 up to 1110 which are briefly described below:
Step 1102—Evaluate whether a state of SpO2 associated with a tissue flap is within a defined range of normal values. If yes, the method proceeds to step 1104, and if no, the method proceeds to generate an alarm at step 1110.
Step 1104—Evaluate whether a detected or measured color state is within a defined range of normal values. If yes, the method proceeds to step 1106, and if no, the method proceeds to generate an alarm at step 1110.
Step 1106—Evaluate whether a detected or measured temperature state is within a defined range of normal values. If yes, the method proceeds to step 1108, and if no, the method proceeds to generate an alarm at step 1110.
Step 1108—Evaluate whether a detected or measured pulse state is within a defined range of normal values. If yes, the state of the tissue flap is determined to be normal and the method ends without generating an alarm, and if no, the method proceeds to generate an alarm at step 1110.

Stated differently, the method of FIG. 11 involves a data processing unit successively (and optionally in the specific order indicated in FIG. 11) evaluating a detected SpO2 value, color value, temperature value and pulse value received from a sensor patch 302 against predefined ranges of acceptable values for each of these data parameters and may trigger an alert or alarm in case any of the individual measured data parameter values exceed their corresponding predefined ranges of acceptable values.

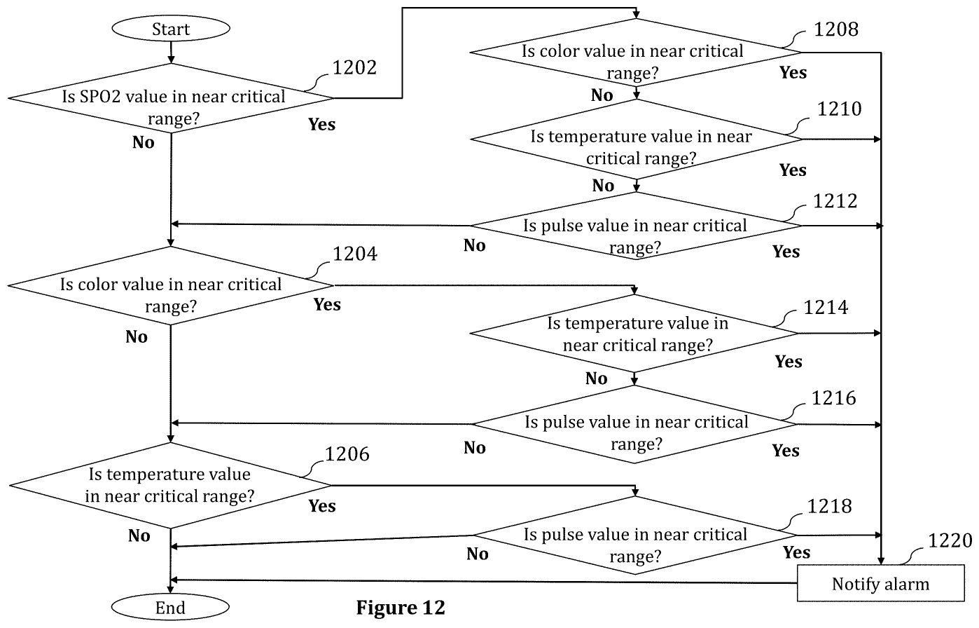

FIG. 12 illustrates an exemplary method of detecting abnormal vascular perfusion based on a combination of data from at least two sensors within a sensor patch 302 located at a site of tissue replantation. The method comprises method steps 1202 up to 1220. The method commences by checking (at step 1202) whether an SpO2 value received from an SpO2 sensor lies outside a predefined range of acceptable SpO2 values—and responsive to determining that the SpO2 data is an outlier data value, checking the data received from the color sensor (at step 1208), temperature sensor (at step 1210) and/or pulse sensor (at step 1212) to see if any of said data values are outside their corresponding predefined ranges of acceptable values as well. In the event any of the color sensor data, temperature sensor data and/or pulse sensor data also comprises outlier data, an alert or alarm may be triggered at step 1220.

If the SpO2 data is not outlier data, the method checks (at step 1204) whether a color value received from a color sensor lies outside a predefined range of acceptable color values—and responsive to determining that the color data is an outlier data value, checking the data received from the temperature sensor (at step 1214) and/or pulse sensor (at step 1216) to see if any of said data values are outside their corresponding predefined ranges of acceptable values as well. In the event any of the temperature sensor data and/or pulse sensor data also comprises outlier data, an alert or alarm may be triggered at step 1220.

If the color data is not outlier data, the method checks (at step 1206) whether a temperature value received from a temperature sensor lies outside a predefined range of acceptable temperature values—and responsive to determining that the temperature data value is an outlier data value, checking the data received from the pulse sensor (at step 12184) to see if the pulse data value is outside a corresponding predefined range of acceptable values as well. In the event the pulse sensor data also comprises outlier data, an alert or alarm may be triggered at step 1220.

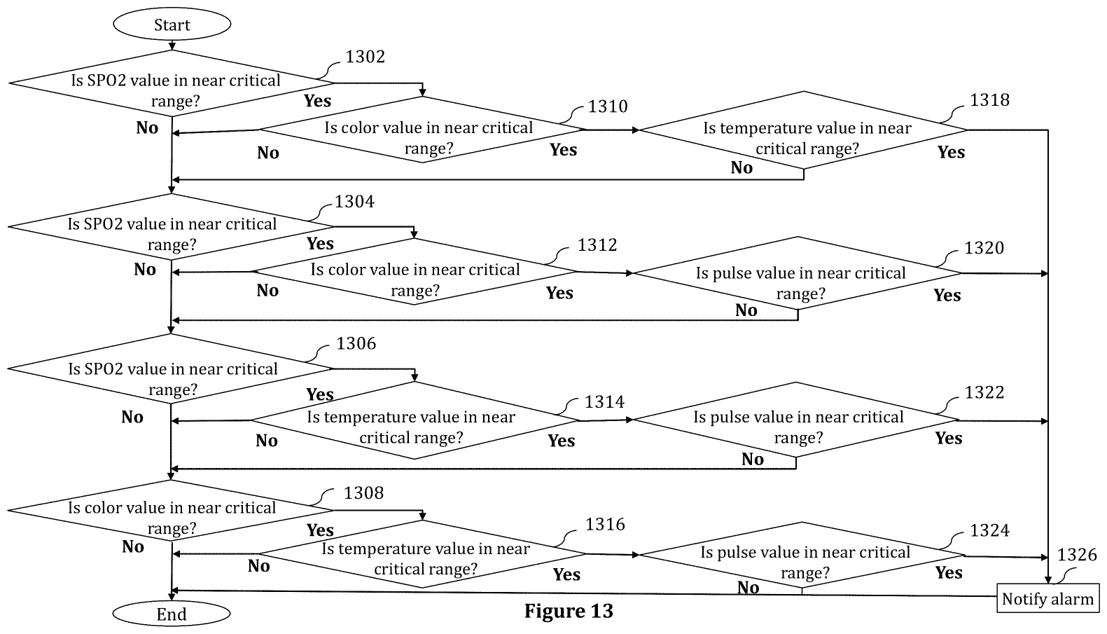

FIG. 13 illustrates an exemplary method of detecting abnormal vascular perfusion based on a combination of data from three sensors within a sensor patch 302 located at a site of tissue replantation. The method comprises method steps 1302 up to 1326. The method commences by checking (at step 1302) whether an SpO2 value received from an SpO2 sensor lies outside a predefined range of acceptable SpO2 values—and responsive to determining that the SpO2 data is an outlier data value, checking (at step 1310) the data received from the color sensor to see if the color data value is outside its corresponding predefined range of acceptable values as well. Responsive to both the SpO2 value and the color value comprising outlier values, the method checks (at step 1318) for whether the received temperature value is outside of its corresponding predefined range of acceptable values. In the event the data received from the SpO2 sensor, color sensor and temperature sensor all comprise outlier values, an alert or alarm may be triggered (at step 1326).

In the event one of the evaluation of SpO2 value (at step 1302), the evaluation of color value (at step 1310) and the evaluation of a temperature value (at step 1318) establishes that said value is within a corresponding predefined range of acceptable values, the method checks (at step 1304, steps 1312 and 1320 respectively) whether a SpO2 value, a color value and a pulse value are all outside their corresponding predefined range of acceptable values. In the event the data received from the SpO2 sensor, color sensor and pulse sensor all comprise outlier values, an alert or alarm may be triggered (at step 1326).

If it is determined (at step 1304, step 1312 and 1320 respectively) that at least one of the data received from the SpO2 sensor, the color sensor and from the pulse sensor is not outlier data, the method checks (at step 1306, steps 1314 and 1322 respectively) data received from the SpO2 sensor, the temperature sensor and from the pulse sensor for whether the received SpO2, temperature and pulse data values are respectively outside of their corresponding predefined range of acceptable values. In the event the data received from the SpO2 sensor, temperature sensor and pulse sensor all comprise outlier values, an alert or alarm may be triggered at step 1326.

If it is determined (at step 1306, step 1314 and 1322 respectively) that at least one of the received data from the SpO2 sensor, temperature sensor and pulse sensor is not an outlier data value, the method checks (at step 1308, 1316 and 1324 respectively) for whether the received color, temperature and pulse data values are respectively outside of their corresponding predefined range of acceptable values. In the event the data received from the color sensor, temperature sensor and pulse sensor all comprise outlier values, an alert or alarm may be triggered at step 1326.

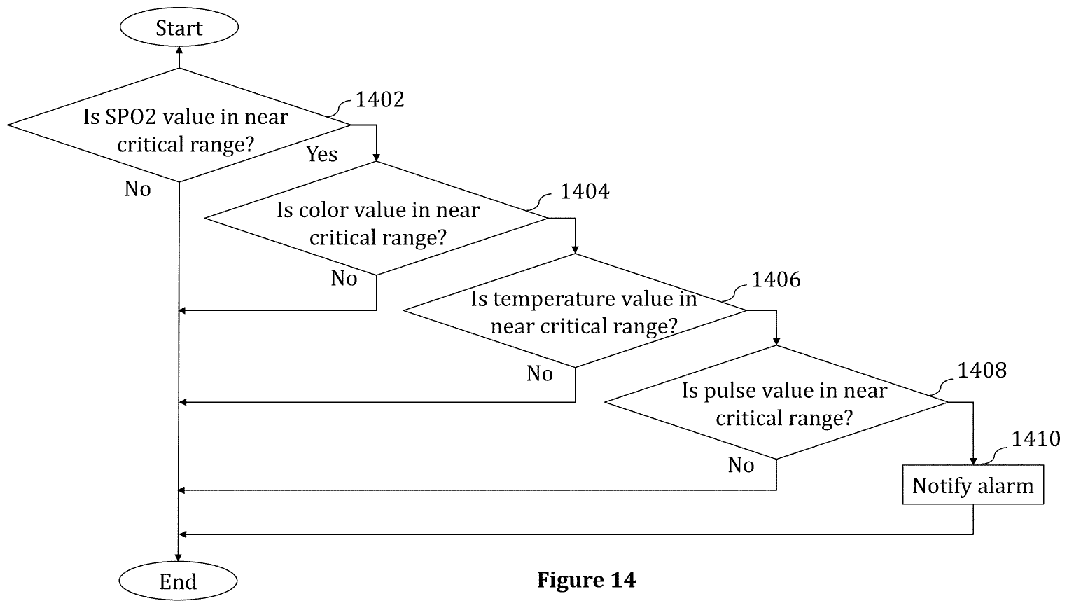

FIG. 14 illustrates an exemplary method of detecting abnormal vascular perfusion based on a combination of data from four sensors within a sensor patch 302 located at a site of tissue replantation. The method comprises method steps 1402 up to 1410.The method comprises checking the SpO2 data (at step 1402), color data (at step 1404), temperature data (at step 1406), and pulse data (at step 1408) values that are received from the corresponding SpO2, color, temperature and pulse sensors within the sensor patch. In the event each of the four data values are respectively outside of their corresponding predefined range of acceptable values, an alert or alarm may be triggered at step 1410.

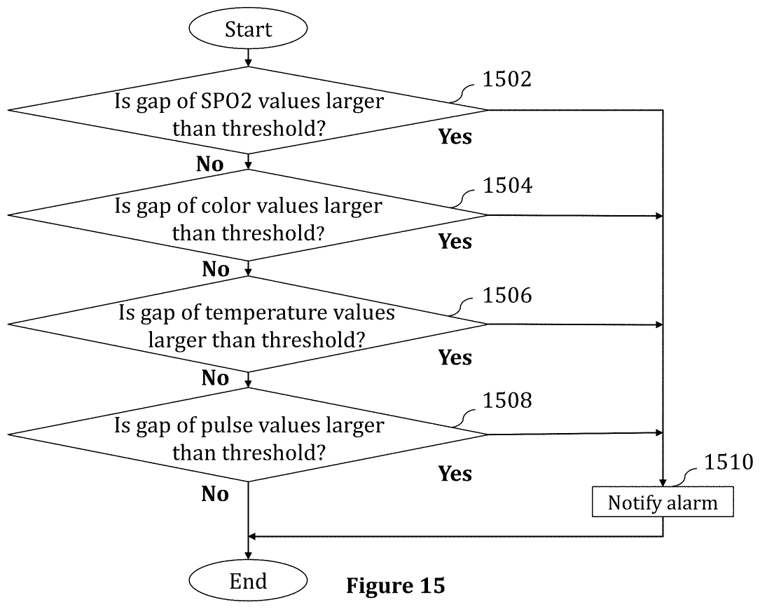

FIG. 15 illustrates a method of a detecting abnormal vascular perfusion based on data received from a first sensor patch 302 (i.e. comprising an SpO2 sensor, a color sensor, a temperature sensor and a pulse sensor) located at a tissue replantation site and a second sensor patch 302′ (i.e. comprising an SpO2 sensor, a color sensor, a temperature sensor and a pulse sensor) located at a healthy tissue site, in accordance with the method of FIG. 6. The method comprises method steps 1502 up to 1510. As shown in FIG. 15, the data processing unit may successively (and optionally in the specific order indicated in FIG. 15) evaluate a difference between the SpO2 values (at step 1502), color values (at step 1504), temperature values (at step 1506)and pulse values (at step 1508)measured at the site of tissue replantation and at a site of healthy tissue, and in the event that a determined difference between the measured two values (for any of the four measurable data parameters) exceeds a predetermined deviation threshold specified in respect of said data parameter, an alert or alarm may be triggered at step 1510.

Since, in several cases, changes in vascular perfusion are very small and not recognizable by unaided human senses (for example, by the unaided eye), problems in vascular perfusion are often difficult to detect and monitor—as a result of which, significant state changes or changes in a vascular perfusion state can be inadvertently overlooked by an operator or healthcare provider.

In an embodiment of the invention, the inventive system and/or method therefore additionally involves magnification of a detected change in data received from sensors, in order to make easy for monitoring the status of the vascular perfusion and for an operator or healthcare provider to recognize and track the changes. The system and/or method of the present invention accordingly magnifies a difference between (i) a predefined baseline value (or range of baseline values) or values received from sensors positioned on healthy tissue and (ii) data received from sensors positioned on the transplanted tissue flap—and the magnified data result is displayed for viewing by users such as doctors, nurses, caregivers etc.

In an embodiment, the magnification may be done based on the following calculation:

Magnified Output=(((Sensor Output)−(Baseline Output))×(Magnification Factor))+(Baseline Value)

The magnification of changes has been found to be especially useful for recognizing or monitoring a change of color—since it has been found to be easier to display and perceive the magnified values visually as the color. However, such magnification techniques can equally be applied to all other sensor data, including data from temperature, SpO2 and/or pulse sensors.

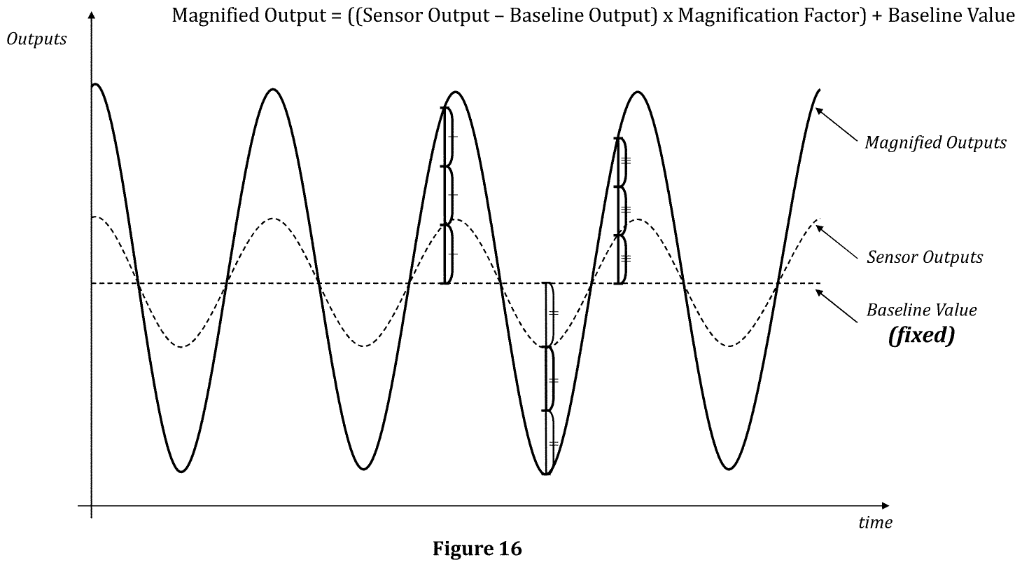

FIG. 16 illustrates a graph corresponding to the output of magnification implemented in accordance with the teachings of the present invention, based on a fixed baseline value. For the data in FIG. 16, it will be understood that (i) the input to the magnification process comprises data received by way of sensor output—i.e. data received from a sensor corresponding to a tissue replantation site, (ii) the fixed baseline value comprises a predefined value or predefined range of values, (iii) the output from the magnification process comprises a determined, calculated or displayed magnified value that is calculated based on sensor output value and a fixed baseline value according to the following equation:

Magnified Output=(((Sensor Output)−(Baseline Output))×(Magnification Factor))+(Baseline Value)

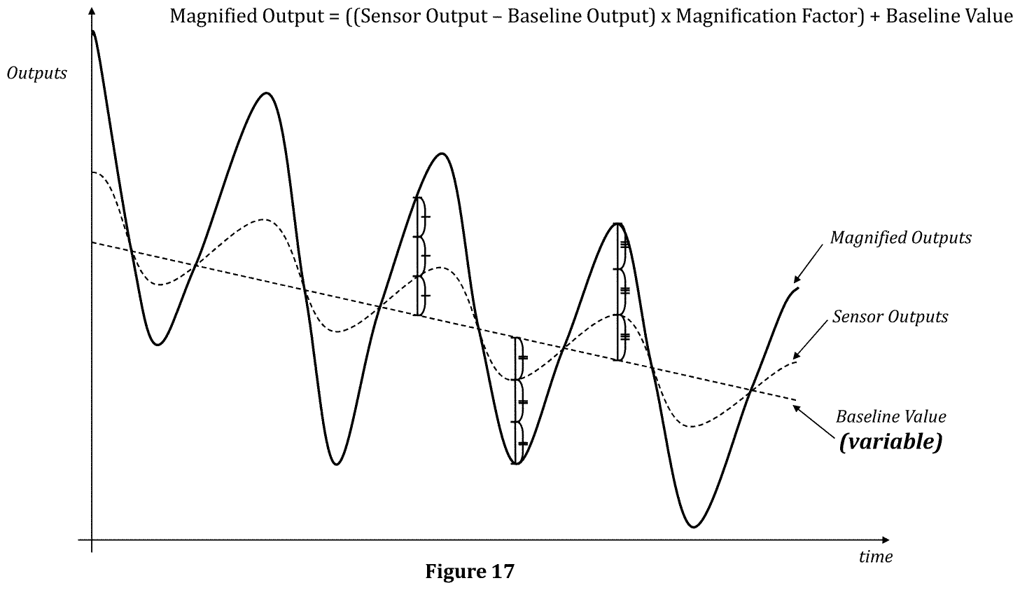

FIG. 17 illustrates a graph corresponding to the output of magnification implemented in accordance with the teachings of the present invention, based on a variable baseline value.

For the data in FIG. 17, it will be understood that (i) the input to the magnification process comprises data received by way of sensor output—i.e. data received from a sensor corresponding to a tissue replantation site, (ii) the variable baseline value comprises a mean value of the received data from a sensor associated with a tissue replantation area or from a sensor associated with healthy tissue or with a value from a sensor associated with healthy tissue, (iii) the output from the magnification process comprises a determined, calculated or displayed magnified value that is calculated based on sensor output value and variable baseline value according to the following equation:

Magnified Output=(((Sensor Output)−(Baseline Output))×(Magnification Factor))+(Baseline Value)

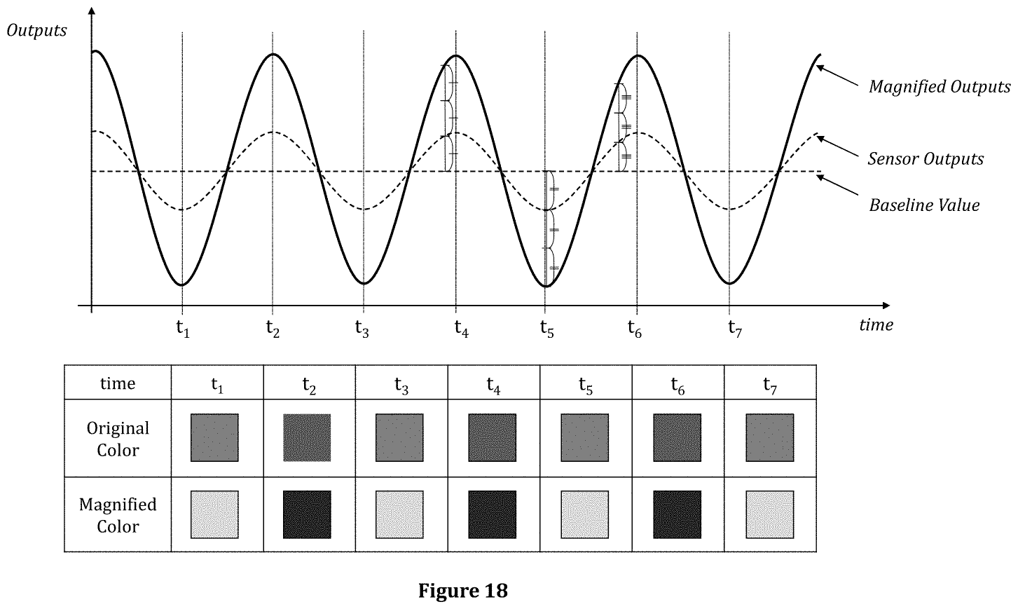

FIG. 18 illustrates a graph corresponding to the output of color magnification implemented in accordance with the teachings of the present invention.

As shown in FIG. 18, the data corresponding to sensor outputs illustrates the colors read by the color sensor, and the data corresponding to the magnified color shows the color outputs calculated by the color magnification. Since the color sensor may originally detect only small differences between tissue color and fixed or variable baseline values, these small differences are difficult to recognize. However, the differences are easily viewed and understood in the magnified color row. Further it will be noted that the colors are changing repeatedly—which indicates that the color sensor can also sense the pulse based on the changes of color on the tissue—and the magnification process makes these changes more distinguishable and readily recognizable.

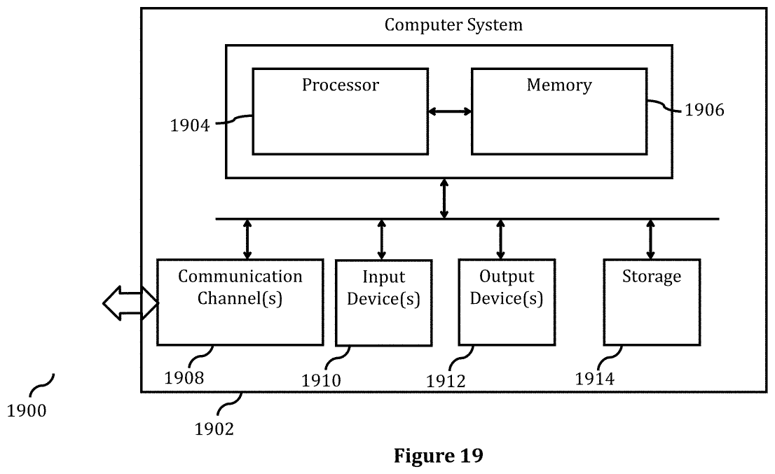

FIG. 19 illustrates an exemplary system 1900 for implementing the present invention.

System 1900 includes computer system 1902 which in turn comprises one or more processors 1904 and at least one memory 1906. Processor 1904 is configured to execute program instructions—and may be a real processor or a virtual processor. It will be understood that computer system 1902 does not suggest any limitation as to scope of use or functionality of described embodiments. The computer system 1902 may include, but is not be limited to, one or more of a general-purpose computer, a programmed microprocessor, a micro-controller, an integrated circuit, and other devices or arrangements of devices that are capable of implementing the steps that constitute the method of the present invention. Exemplary embodiments of a computer system 1902 in accordance with the present invention may include one or more servers, desktops, laptops, tablets, smart phones, mobile phones, mobile communication devices, tablets, phablets and personal digital assistants. In an embodiment of the present invention, the memory 1906 may store software for implementing various embodiments of the present invention. The computer system 1902 may have additional components. For example, the computer system 1902 may include one or more communication channels 1908, one or more input devices 1910, one or more output devices 1912, and storage 1914. An interconnection mechanism (not shown) such as a bus, controller, or network, interconnects the components of the computer system 1902. In various embodiments of the present invention, operating system software (not shown) provides an operating environment for various softwares executing in the computer system 1902 using a processor 1904, and manages different functionalities of the components of the computer system 1902.

The communication channel(s) 1908 allow communication over a communication medium to various other computing entities. The communication medium provides information such as program instructions, or other data in a communication media. The communication media includes, but is not limited to, wired or wireless methodologies implemented with an electrical, optical, RF, infrared, acoustic, microwave, Bluetooth or other transmission media.

The input device(s) 1910 may include, but is not limited to, a touch screen, a keyboard, mouse, pen, joystick, trackball, a voice device, a scanning device, or any another device that is capable of providing input to the computer system 1902. In an embodiment of the present invention, the input device(s) 1910 may be a sound card or similar device that accepts audio input in analog or digital form. The output device(s) 1912 may include, but not be limited to, a user interface on CRT, LCD, LED display, or any other display associated with any of servers, desktops, laptops, tablets, smart phones, mobile phones, mobile communication devices, tablets, phablets and personal digital assistants, printer, speaker, CD/DVD writer, or any other device that provides output from the computer system 1902.

The storage 1914 may include, but not be limited to, magnetic disks, magnetic tapes, CD-ROMs, CD-RWs, DVDs, any types of computer memory, magnetic stripes, smart cards, printed barcodes or any other transitory or non-transitory medium which can be used to store information and can be accessed by the computer system 1902. In various embodiments of the present invention, the storage 1914 may contain program instructions for implementing any of the described embodiments.

In an embodiment of the present invention, the computer system 1902 is part of a distributed network or a part of a set of available cloud resources.

The present invention may be implemented in numerous ways including as a system, a method, or a computer program product such as a computer readable storage medium or a computer network wherein programming instructions are communicated from a remote location.

The present invention may suitably be embodied as a computer program product for use with the computer system 1902. The method described herein is typically implemented as a computer program product, comprising a set of program instructions that is executed by the computer system 1902 or any other similar device. The set of program instructions may be a series of computer readable codes stored on a tangible medium, such as a computer readable storage medium (storage 1914), for example, diskette, CD-ROM, ROM, flash drives or hard disk, or transmittable to the computer system 1902, via a modem or other interface device, over either a tangible medium, including but not limited to optical or analogue communications channel(s) 1908. The implementation of the invention as a computer program product may be in an intangible form using wireless techniques, including but not limited to microwave, infrared, Bluetooth or other transmission techniques. These instructions can be preloaded into a system or recorded on a storage medium such as a CD-ROM, or made available for downloading over a network such as the Internet or a mobile telephone network. The series of computer readable instructions may embody all or part of the functionality previously described herein.

Based on the above it would be understood that the present invention offers significant advantages over prior art solutions, in terms of enabling sensor based, monitoring of vascular perfusion in replanted tissue flaps, which is non-invasive, does not subject the replanted tissue to additional trauma during blood sample collection, and provides continuous real-time results and evaluations of replanted tissue flap health, and that is capable of detecting problems in vascular perfusion and raising alarms in real time.

While the exemplary embodiments of the present invention are described and illustrated herein, it will be appreciated that they are merely illustrative. It will be understood by those skilled in the art that various modifications in form and detail may be made therein without departing from or offending the spirit and scope of the invention as defined by the appended claims. Additionally, the invention illustratively disclose herein suitably may be practiced in the absence of any element which is not specifically disclosed herein—and in a particular embodiment that is specifically contemplated, the invention is intended to be practiced in the absence of any one or more element which are not specifically disclosed herein.

1. An apparatus for non-invasive monitoring of vascular perfusion at a site of tissue replantation, comprising:
a processor;
a transceiver;
one or more of a first temperature sensor, a first SpO2 sensor, a first pulse sensor and a first color sensor;
wherein the processor is configured to:
receive from one or more of a first temperature sensor, a first SpO₂ sensor, a first pulse sensor and a first color sensor respectively, one or more of temperature data, SpO₂ data, pulse data and color data corresponding to a tissue replantation site; and
evaluate whether any of the received temperature data, SpO₂ data, pulse data and color data received, that any of a detected temperature parameter value, SpO₂ parameter value, pulse parameter value and color parameter value corresponding to the tissue replantation site, is consistent with one or more range of normal values for temperature data, SpO₂ data, pulse data or color data.

2. The apparatus as claimed in claim 1, configured such that the one or more normal values for temperature data, SpO₂ data, pulse data or color data comprise a range of predefined normal values associated with healthy tissue.

3. The apparatus as claimed in claim 1, wherein the one or more normal values for temperature data, SpO₂ data, pulse data or color data comprise one or more of temperature data values received from a second temperature sensor, SpO₂ data received from a second SpO₂ sensor, pulse data received from a second pulse sensor and color data received from a second color sensor, wherein any of the second temperature sensor, second SpO₂ sensor, second pulse sensor, and second color sensor are configured to detect data values corresponding to a healthy tissue site.

4. The apparatus as claimed in claim 1, comprising all of a first temperature sensor, a first SpO₂ sensor, a first pulse sensor and a first color sensor.

5. The apparatus as claimed in claim 16, configured such that the alarm is generated based on an evaluation of two or more of temperature data, SpO₂ data, pulse data and color data corresponding to the tissue replantation site.

6. The apparatus as claimed in claim 5, configured such that the alarm is generated based on an evaluation of all of temperature data, SpO₂ data, pulse data and color data corresponding to the tissue replantation site.

7. The apparatus as claimed in claim 1, configured such that data received from any of the first temperature sensor, first SpO₂ sensor, first pulse sensor and first color sensor respectively is magnified for display, wherein a magnified output is determined as:
Magnified Output=(((Sensor Output)−(Baseline Output))×(Magnification Factor))+(Baseline Value).

8. A method for non-invasive monitoring of vascular perfusion at a site of tissue replantation, comprising implementing at a processor, the steps of:
receiving from one or more of a first temperature sensor, a first SpO₂ sensor, a first pulse sensor and a first color sensor respectively, one or more of temperature data, SpO₂ data, pulse data and color data corresponding to a tissue replantation site; and
evaluate whether any of the received temperature data, SpO₂ data, pulse data and color data received, that any of a detected temperature parameter value, SpO₂ parameter value, pulse parameter value and color parameter value corresponding to the tissue replantation site, is consistent with one or more range of normal values for temperature data, SpO₂ data, pulse data or color data.

9. The method as claimed in claim 8, wherein said one or more normal values comprising a range of predefined normal values associated with healthy tissue.

10. The method as claimed in claim 8, wherein the one or more normal values for temperature data, SpO₂ data, pulse data or color data comprise one or more of temperature data values received from a second temperature sensor, SpO₂ data received from a second SpO₂ sensor, pulse data received from a second pulse sensor and color data received from a second color sensor, wherein any of the second temperature sensor, second SpO₂ sensor, second pulse sensor, and second color sensor are configured to detect data values corresponding to a healthy tissue site.

11. The method as claimed in claim 8, comprising receiving all of temperature data, SpO₂ data, pulse data and color data corresponding to a tissue replantation site.

12. The method as claimed in claim 18, wherein the alarm is generated based on an evaluation of two or more of temperature data, SpO₂ data, pulse data and color data corresponding to the tissue replantation site.

13. The method as claimed in claim 12, wherein the alarm is generated based on an evaluation of all of temperature data, SpO₂ data, pulse data and color data corresponding to the tissue replantation site.

14. The method as claimed in claim 8, wherein data received from any of the first temperature sensor, first SpO₂ sensor, first pulse sensor and first color sensor respectively is magnified for display, wherein a magnified output is determined as:
Magnified Output=(((Sensor Output)−(Baseline Output))×(Magnification Factor))+(Baseline Value).

15. A computer program product for non-invasive monitoring of vascular perfusion at a site of tissue replantation, comprising a non-transitory computer readable medium having computer readable instructions for implementing the steps of:
receiving from one or more of a first temperature sensor, a first SpO₂ sensor, a first pulse sensor and a first color sensor respectively, one or more of temperature data, SpO₂ data, pulse data and color data corresponding to a tissue replantation site; and
evaluate whether any of the received temperature data, SpO₂ data, pulse data and color data received, that any of a detected temperature parameter value, SpO₂ parameter value, pulse parameter value and color parameter value corresponding to the tissue replantation site, is consistent with one or more range of normal values for temperature data, SpO₂ data, pulse data or color data.

16. The apparatus as claimed in claim 1, wherein the processor generates an alarm in response to determining, based on any of the received temperature data, SpO2 data, pulse data and color data received, that any of a detected temperature parameter value, SpO2 parameter value, pulse parameter value and color parameter value corresponding to the tissue replantation site, is inconsistent with one or more normal values for temperature data, SpO2 data, pulse data or color data.

17. The apparatus as claimed in claim 1, wherein the processor continuously receives one or more of temperature data, SpO2 data, pulse data and color data corresponding to a tissue replantation site.

18. The method as claimed in claim 8, wherein the processor generates an alarm in response to determining, based on any of the received temperature data, SpO2 data, pulse data and color data received, that any of a detected temperature parameter value, SpO2 parameter value, pulse parameter value and color parameter value corresponding to the tissue replantation site, is inconsistent with one or more normal values for temperature data, SpO2 data, pulse data or color data.

19. The method as claimed in claim 8, wherein the processor continuously receives one or more of temperature data, SpO2 data, pulse data and color data corresponding to a tissue replantation site.
